(19) United States
(12) Patent Application Publication
(10) Pub. No.: US 2012/0214820 A1
(43) Pub. Date: Aug. 23, 2012

(54) ORALLY DISINTEGRATING PHARMACEUTICAL DOSAGE FORM CONTAINING ARIPIPRAZOLE

(75) Inventors: Parshwakumar V. Kalyankar, Aurangabad (IN); Ganesh V. Gat, Pune (IN); Javed Hussain, Aurangabad (IN)

(73) Assignee: ratiopharm GmbH, Ulm (DE)

(21) Appl. No.: 13/496,356

(22) PCT Filed: Sep. 9, 2010
(86) PCT No.: PCT/EP10/63226

§ 371 Date: May 8, 2012

(30) Foreign Application Priority Data
Sep. 15, 2009 (IN) 1916/DEL/2009

(51) Int. Cl. A61K 31/496; B29C 43/02; A61P 25/22; A61P 25/18; A61P 25/24

(52) U.S. Cl. 514/253.07; 264/122

(57) ABSTRACT

The present invention relates to an orally disintegrating dosage form containing aripiprazole. The formulation of the invention shows a fast disintegration.

### FIELD OF THE INVENTION

The present invention is directed to an orally disintegrating dosage form containing aripiprazole. More precisely the present invention is directed to a dosage form containing aripiprazole that disintegrates in the oral cavity within about 20 to about 30 seconds or less.

### BACKGROUND OF THE INVENTION

Aripiprazole is an atypical antipsychotic agent useful for the treatment of schizophrenia (EP 0 367 141). Further investigations revealed that aripiprazole is also effective in the treatment of acute bipolar mania (Paul E. Keck, Am. J. Psychiatry 2003, 160, 1651-1658). It was also reported that aripiprazole may be effective as an augmentation for patients with persistent depressive and anxiety disorders despite initial treatment with selective serotonin reuptake inhibitors (John J. Worthington et al., International Clinical Psychopharmacology, 2005, 1, pages 9-11). The structure of aripiprazole is as follows:

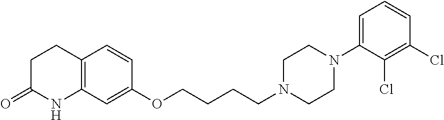

Aripiprazole is a polymorphic substance. Aoki et al. describe two anhydrous aripiprazole crystal forms type I and type II and a hydrate form (proceedings of the 4^{th} Japanese-Korean Symposium on Separation Technology, Tokyo, Oct. 6-8, 1996). WO 03/026659 A1 and EP 1 330 249 B1 describe different polymorphic forms of aripiprazole, including “anhydrous crystals B” and “hydrate A”.

EP 1 808 164 A1/WO 2007/081366 A1 and EP 1 808 165 A1/WO 2007/081367 disclose wet granulation formulations of aripiprazole and dry formulations of aripiprazole, respectively. These applications disclose a dissolution rate of the manufactured tablets of not less than 85-95% by weight of the initial aripiprazole after 30 minutes. The compositions correspond to the original immediate release (IR) tablets Abilify®. Therefore, the teaching of these patent applications does relate to IR dosage forms.

There are a number of varieties of solid pharmaceutical dosage forms that rapidly dissolve or disintegrate in a glass of water, in the mouth or in the gastrointestinal tract. The advantages of dosage forms disintegrating in the mouth are an immediate administration of a drug and also a convenient way of administration in situations where no water is available to dissolve a tablet or to ease swallowing of a tablet. Therefore, the use of orally disintegrating dosage forms for the administration of medicaments especially helps patients who are very young, elder, non-compliant or have a physical impairment which makes it difficult for them to swallow an intact dosage form.

An important function of an orally disintegrating dosage form is to allow a quick absorption of a therapeutically active compound, which is achieved by a short disintegration time of the dosage form in the oral cavity. Due to the requirement that orally disintegrating dosage forms should resolve quickly in the mouth, the dosage forms are generally highly hygroscopic and susceptible to moisture. Therefore, special precautions in the preparation, packaging, handling and storing of the dosage form are to be taken. Susceptibility to water and moisture is an inherent side effect of such preparations. Attempts to decrease susceptibility to water therefore often result in an increase of the disintegration time. The potential problems due to hygroscopicity of the dosage forms are thus left aside as there is still need for rapidly disintegrating dosage forms to ensure fast absorption of a medicament in a convenient way.

In order to achieve a short disintegration time, the orally disintegrating dosage forms are furthermore often highly porous resulting in a decreased mechanical stability. European patent EP 1 145 711 discloses an aripiprazole containing orally disintegrating dosage form exhibiting a satisfactory stability. The main ingredient of that dosage form is calcium silicate (about 35 wt.-%). Calcium silicate is known to be poorly compressible due to its fluffiness. Therefore, the forces required for tablet compression will be relatively high due to the high amount of calcium silicate in the formulation. Furthermore, higher compression forces can result in an increase of the disintegration time of the tablets.

The above mentioned WO 03/026659 also discloses flash-melt formulations of the “hydrate A” and the “anhydrous crystals B” of aripiprazole described therein. In these formulations calcium silicate is the main excipient.

An object of the present invention is therefore to provide an orally disintegrating dosage form which shows a better performance regarding the above-mentioned properties, the disintegration time being of particular relevance.

The inventors surprisingly found that compressibility and even the disintegration time can be improved when the high functionality excipient silicified microcrystalline cellulose is used as the main ingredient of the orally disintegrating dosage form containing aripiprazole. The present invention thus solves the above-mentioned problem by providing an orally disintegrating dosage form, a process and a use according to the present claims.

### SUMMARY OF THE INVENTION

The present invention provides an orally disintegrating pharmaceutical dosage form comprising aripiprazole and 10-70% of silicified microcrystalline cellulose (SMC) based on the total weight of the dosage form.

In one embodiment of the present invention the orally disintegrating pharmaceutical dosage form further comprises 10-50% of a filler, 0.5-5% of a binder, 5-35% of a disintegrant and 0.5-5% of a distributing agent, based on the total weight of the dosage form.

In another embodiment, the orally disintegrating pharmaceutical dosage form comprises 20-65% of SMC, 15-40% of a filler, 1-3% of a binder, 8-30% of a disintegrant and 1-4% of a distributing agent based on the total weight of the dosage form.

In another embodiment, the filler of the orally disintegrating pharmaceutical dosage form is microcrystalline cellulose, starch; lactose and/or mannitol.

In another embodiment, the binder of the orally disintegrating pharmaceutical dosage form is hydroxypropyl cellulose and/or ethylcellulose.

In another embodiment the disintegrant of the orally disintegrating pharmaceutical dosage form is crospovidone, carmellose, croscarmellose sodium and/or sodium starch glycolate.

In another embodiment, the distributing agent of the orally disintegrating pharmaceutical dosage form is colloidal SiO₂, fumed silica, diatomaceous earth, kaolin, talc, and/or magnesium aluminium trisilicate.

In a preferred embodiment of the present invention the filler of the orally disintegrating pharmaceutical dosage form is microcrystalline cellulose, the binder is hydroxypropyl cellulose, the disintegrant is crospovidone and/or carmellose and the distributing agent is colloidal SiO₂.

In another preferred embodiment, the orally disintegrating pharmaceutical dosage form is a tablet.

In another embodiment of the present invention aripiprazole is present preferably in an amount in the range of 2-30% based on the total weight of the dosage form.

In another preferred embodiment, the aripiprazole is present in the orally disintegrating pharmaceutical dosage form in the form of anhydrous aripiprazole.

In another preferred embodiment, the aripiprazole is present in the orally disintegrating pharmaceutical dosage form in the form of hygroscopic aripiprazole.

Another aspect of this invention is an orally disintegrating pharmaceutical dosage form comprising aripiprazole which disintegrates within 20 seconds or less, determined according to USP 29, <701> Disintegration, pp. 2670-2672.

The present invention further provides a process for the manufacture of an orally disintegrating pharmaceutical dosage form containing aripiprazole comprising the steps of
i) blending aripiprazole with SMC and at least one further excipient,
ii) granulating the blend obtained in step i),
iii) blending the granules with at least one further excipient comprising a disintegrant and
iv) compressing the granules to a tablet.

In a preferred embodiment of the process according to the present invention, the granulation step ii) comprises wet granulation followed by a drying step.

In another embodiment of the process, the wet granulation is carried out with water or an aqueous binder solution and the drying is carried out above 70° C.

Preferably the process according to the present invention comprises the steps of dry blending aripiprazole with SMC, dry blending a filler with a distributing agent, wet granulating said blends together with a binder solution of hydroxypropyl cellulose in water, drying above 70% and adding at least one further excipient comprising a disintegrant.

In another preferred embodiment of the process of the present invention, the granulation step of ii) comprises dry granulation.

Furthermore, the present invention provides an orally disintegrating pharmaceutical dosage form obtainable by a process according to any embodiment of the process of the present invention.

Furthermore, the present invention provides the use of SMC for the manufacture of an orally disintegrating pharmaceutical dosage form containing aripiprazole.

Furthermore, the present invention provides the use of aripiprazole for the manufacture of an orally disintegrating pharmaceutical dosage form containing SMC.

The present invention further provides an orally disintegrating pharmaceutical dosage form for the treatment of schizophrenia, acute mania, depression, anxiety, bipolar disorder and mixed episodes associated with bipolar disorder.

### DETAILED DESCRIPTION OF THE INVENTION

Disintegration Properties

The phrase “orally disintegrating dosage form” as used herein denotes a solid oral preparation containing at least one active agent which disintegrates rapidly in the oral cavity, with an in vitro disintegration time of 60 seconds or less, when based on the United States Pharmacopeia (USP) disintegration test method (USP 29, <701> Disintegration, pp. 2670-2672). The phrase “orally disintegrating dosage form” includes solid orally dispersible dosage forms or flashmelt dosage forms and is hereinafter abbreviated with “ODT” (“Oral Disintegrating Tablet”). The form of the ODT of the present invention is not limited to a tablet, but can either be a caplet, a wafer, pellets, a capsule, a pill, a sachet, a powder or a granulate and the like.

The ODT according to the present invention exhibits very favourable disintegration properties. The disintegration time of the ODT of the present invention is preferably 30 seconds or less, more preferably 25 seconds or less, still more preferably 20 seconds or less, most preferably 15 seconds or less, determined according to USP 29, <701> Disintegration, pp. 2670-2672.

Aripiprazole

Aripiprazole can be present in any form. For instance, aripiprazole can be present in anhydrous form which can be either hygroscopic or non-hygroscopic. Furthermore, it can be present in its hydrate form. The ODT of the present invention preferably comprises anhydrous aripiprazole. In one embodiment, the ODT of the present invention comprises hygroscopic anhydrous aripiprazole. In another embodiment, the ODT of the present invention comprises non-hygroscopic anhydrous aripiprazole.

In particular, it is referred to the above mentioned crystal forms published by Aoki et al. In a special embodiment of the present invention anhydrous crystal type I according to Aoki et al. is present in the ODT.

In another special embodiment of the present invention an anhydrous crystal type II according to Aoki et al. is present in the ODT. In still another special embodiment of the present invention hydrous crystals according to Aoki et al. is present in the ODT.

In all embodiments of the present invention, the possible aripiprazole forms can be used for the preparation of the ODT of the present invention and/or be present in the readily prepared ODT of the present invention.

The relative amount of aripiprazole present in the ODT according to the invention is not particularly limited. Preferably, aripiprazole is present in the range of 2-30% by weight, preferably 3 to 20% by weight, more preferably 4 to 15% by weight, most preferably from 5 to 10% by weight based upon the total weight of the dosage form. Unless indicated otherwise, percentages are to be understood as meaning “% by weight” in this application.

Generally, according to the present invention the ODT may contain from 2 mg to 50 mg of aripiprazole. Preferred amounts are 5, 10, 15, 20 and 30 mg of aripiprazole.

To obtain the favourable disintegration properties described supra, the ODT of this invention preferably comprises a high functionality excipient which is silicified microcrystalline cellulose (SMC), a filler, a disintegrant and a distributing agent.

Silicified Microcrystalline Cellulose

The inventors found that “silicified microcrystalline cellulose” (SMC) is an ideal excipient in combination with aripiprazole over the whole range of the amount of aripiprazole present in the dosage form.

SMC is composed of intimately associated microcrystalline cellulose and colloidal silicon dioxide particles, derived from aqueous coprocessing prior to drying the material during manufacture. The microcrystalline cellulose component is purified, partially depolymerized cellulose, prepared by treating alpha cellulose, obtained as a pulp from fibrous plant material, with mineral acids. The colloidal silicon dioxide is a submicroscopic fumed silica prepared by the vaporphase hydrolysis of a silicon compound.

The preparation and characteristics of SMC are disclosed in U.S. Pat. No. 5,585,115 or WO 96/21429. SMC has significantly improved properties in view of compressibility, disintegration time and others compared to a simple blend of microcrystalline cellulose and colloidal silicon dioxide. These differences are based on a unique preparation method of special predesigned ingredients.

The particle size of silicon dioxide for example used for the preparation of SMC is of particular relevance. The average primary particle diameter of the preferred class of silicon dioxides utilized in the preparation of SMC ranges from about 5 nm to about 50 nm. The surface area of the preferred class of silicon dioxides utilized in the preparation of SMC ranges from about 50 m²/g to about 500 m²/g.

The relative amount of microcrystalline cellulose to colloidal silicon dioxide is also of relevance. Colloidal silicon dioxide is present in an amount of from about 0.5% to about 10%, preferably it is present in an amount of about 2 wt.-% based on the amount of microcrystalline cellulose.

The advantageous properties of SMC are due to the preparation process. In this process an aqueous slurry of microcrystalline cellulose and colloidal silicon dioxide is subjected to spray-drying after a uniform mixture of the ingredients is obtained in the suspension. In the spray-drying process, the aqueous dispersion of microcrystalline cellulose and silicon dioxide is brought together with a sufficient volume of hot air to produce evaporating and drying of the liquid droplets. The highly dispersed slurry of microcrystalline cellulose and silicon dioxide is pumpable and capable of being atomized. It is sprayed into a current of warm filtered air, which supplies the heat for evaporating and conveys a dried product to a collective device. The air is then exhausted with the removed moisture. The resulted spray-dried powder particles are approximately spherical in shape and are relatively uniform in size, thereby possessing excellent flowability. The co-processed product consists of microcrystalline cellulose and silicon dioxide in intimate association with each other. Magnifications of the resulted particles indicate that the silicon dioxide is integrated with or partially coats the surfaces of the microcrystalline cellulose particles. These particles possess desirable performance attributes that are not present when microcrystalline cellulose and silicon dioxide are combined in a dry mixture. It is believed that the beneficial result obtained by the combination of these two materials is due to the fact that the two materials are intimately associated with each other. Regarding the properties and methods of preparation of SMC, the whole disclosure of U.S. Pat. No. 5,585,115 is incorporated herein by reference.

SMC is to be understood as an excipient on its own, having different properties compared to a simple blend of microcrystalline cellulose and colloidal silicon dioxide. Therefore, it is possible that microcrystalline cellulose and colloidal silicon dioxide can additionally be used in the ODT of the present invention. Thus, any disclosed amount of microcrystalline cellulose or colloidal SiO₂ present in the ODT of the present invention does not include the amount of microcrystalline cellulose or colloidal SiO₂ present in SMC.

Further Excipients

Preferably a binder is also present in the formulation. The binder, if any, present in the ODT of the present invention can be for example hydroxypropylcellulose, ethylcelluloSe and the like. Preferably the binder is hydroxypropylcellulose. Preferably the binder is present in an amount of 0.5-5%, more preferably 1-3% based on the total weight of the ODT of the present invention.

The disintegrant present in the ODT of the present invention can be for example crospovidone, carmellose, croscarmellose sodium, sodium starch glycolate and the like. Preferred disintegrants are crospovidone and carmellose. Preferably the disintegrant is present in an amount of 5-35%, more preferably 8-30%, even more preferably 10-25% and most preferably about 15% based on the total weight of the ODT of the present invention.

Microcrystalline cellulose can be used as a filler in the ODT of the present invention. The fillers used in the present invention are not particularly limited and include also lactose, mannitol, sorbitol and the like. Preferably the filler is microcrystalline cellulose. The filler is preferably present in an amount of 10-50%, more preferably 15-45%, even more preferably 20-40% and most preferably 25-35% based on the total weight of the ODT.

Colloidal silicon dioxide can be used as a distributing agent or glidant in the ODT of the present invention. The distributing agent present in the ODT of the present invention is not particularly limited to colloidal SiO₂. Other distributing agents well-known to a person skilled in the art can also be present in the ODT of the present invention. Preferably the distributing agent is present in an amount in the range of 1-4 wt.-% based on the total weight of the ODT. In one preferred embodiment of the ODT of the present invention, the distributing agent is present in an amount of about 2 wt.-% based on the total weight of the ODT.

The combination of excipients may be formulated with other conventional adjuncts, particularly flavouring agents, flavour enhancers, sweetening agents, lubricants and the like, which are also well-known in the art. These include, for example, but are not limited to natural and artificial flavours, natural sweetening agents like polyols such as mannitol, sorbitol, maltitol and xylitol, articifial sweetening agents such as aspartame and acesulfame K, flavour enhancers such as tartaric acid and lubricants such as magnesium stearate, starch, talc and the like. It is well-known to the person skilled the art that the amount of flavouring and sweetening agents, if any, present in the formulations of the present invention will be directly dependent on the taste or bitterness and the amount of the therapeutically active ingredient, i.e. aripiprazole. The flavouring and sweetening agents do not serve to coat the ODT or the medicament, but are adequate to mask the objectionable taste of aripiprazole in admixture therewith. In general, the total of such conventional adjuncts will not exceed 35%, preferably being in a range of 20-30% by weight based on the total weight of the ODT.

Method of Preparation

In a further aspect, the present invention provides a process for the manufacture of an orally disintegrating pharmaceutical dosage form containing aripiprazole said process comprising the following steps:
i) blending aripiprazole with SMC and at least one further excipient,
ii) granulating the blend obtained in step i)
iii) blending the granules with at least one further excipient comprising a disintegrant and
iv) compressing the blend to a tablet.

When wet granulation is performed, the wet granules obtained from step ii) are usually dried and sized prior to step iii), see infra.

In some passages of the present invention it is referred to an intragranulation part or an extragranulation part of the process of the invention. The intragranulation comprises the steps i) and ii) and the extragranulation comprises the steps iii) and iv). This means that any action of the process carried out before the granulation step is finished belongs to the intragranulation and any action performed after the granulation step is finished belongs to the extragranulation.

In the intragranulation part of the process, the active ingredient aripiprazole, SMC and preferably the filler and the glidant are blended together. More preferably also a binder is added to the composition during the intragranulation part of the process.

It is also possible that one or more ingredients of the intragranulation part of the process are added during the granulation step ii) of the process.

Furthermore, it is also possible that further adjuncts are added in the intragranulation step, like e.g. sweetening agents, flavour enhancers or flavour. Preferably, however, said adjuncts are added in the extragranular part of the process.

In one embodiment of the process of the present invention, the granulation step ii) comprises wet granulation. Preferably the intragranular blend is subjected to wet granulation with water, preferably purified water, or with a binder solution, preferably hydroxypropyl cellulose in water, preferably purified water.

When the wet granulation is completed, the material is dried, preferably at a temperature above 70° C., more preferably in the range of 70 to 95° C., even more preferably in the range of 75 to 90° C., most preferably about 80° C. The drying time depends on the total amount of the granules. Usually, drying is carried out until the loss of weight of the granules reaches 1.5 to 2.5 %.

In another embodiment of the process of the present invention, the intragranulation part comprises dry granulation instead of wet granulation. In this embodiment the blend of aripiprazole and SMC and preferably the filler, the glidant and the binder is subjected to dry granulation.

After the intragranulation part of the process, the obtained material is then subjected to the extragranulation part of the process. In the extragranulation part of the process, the material obtained from the intragranulation part is mixed with at least one further excipient comprising the disintegrant. Preferably other adjuncts like e.g. sweetening agents, flavour enhancers, flavours and lubricants are also added. After the mixing or blending of all ingredients in the extragranulation is completed, the blend is compressed to a tablet.

In still another aspect of the present invention, a process is provided, wherein aripiprazole and the aforementioned ingredients are subjected to direct compression.

In all embodiments of the process of the present invention, all solid ingredients are preferably milled and sieved before being used in the process of the present invention. It is also possible that milling and sieving can be applied on the ingredients or blends before and after any step of the process of the present invention. Preferably the material obtained after the granulation step, regardless to whether wet granulation or dry granulation is carried out, is subjected to milling and sieving. The milled ingredients or blends or granules are preferably sieved through ASTM No. 30 or No. 40 sieves, most preferably through an ASTM No. 40 sieve.

In a preferred embodiment of the process of the present invention, aripiprazole is mixed with SMC and the blend is sieved through an ASTM No. 40 sieve. Microcrystalline cellulose and colloidal silicon dioxide are mixed separately and sieved through an ASTM No. 40 sieve. A binder solution is prepared by adding hydroxypropyl cellulose to water during continuous stirring. The aforesaid dry blends are then mixed together in a high shear mixer granulator (HSMG). The mixture obtained therefrom is then subjected to wet granulation with said binder solution, i.e. aqueous hydroxypropyl cellulose solution. The material obtained after the wet granulation is then dried with a rapid dryer (e.g. at 75 to 85° C.), milled, and sieved through an ASTM No. 30 sieve. These granules are again milled and sieved through an ASTM No. 40 sieve. Carmellose, crospovidone, xylitol, aspartame, acesulfame potassium, tartaric acid, and a flavour are separately mixed together, milled and sieved through an ASTM No. 40 sieve. The resulting blend is brought together with the aforesaid granules and mixed well followed by additional sieving through an ASTM No. 40 sieve. Magnesium stearate is added to the mixture and the blend is mixed again well. Finally, the obtained blend is compressed, e.g. by using a 7.1 mm round, flat faced with beveled edged having breakline on upper punch and lower punch plain.

The respective relative amounts of aripiprazole and the other ingredients comply with those present in the ODT of the present invention mentioned above.

According to another aspect of the present invention, an ODT obtainable from the process of the present invention is provided.

Further Properties of the ODT of the Invention

The weight of the readily obtained ODT is not particularly limited. Preferably the total weight of the tablet is from 100 to 200 mg, e.g. about 150 mg.

The ODTs of the present invention have sufficient mechanical stability. Preferably, they have a hardness of 40 to 70 N, preferably 50 to 60 N, determined according to Pharm. Eur. 6.0 <2.9.8>. This hardness of the ODT of the invention is another advantage of the present invention.

Preferably the ODT of the present invention does not contain calcium silicate because of the poor compressibility of this material.

Yet another aspect of the present invention provides the use of SMC for the manufacture of an orally disintegrating pharmaceutical dosage form containing aripiprazole.

Yet another embodiment of the present invention provides the use of aripiprazole for the manufacture of an orally disintegrating pharmaceutical dosage form containing SMC.

The ODT of the present invention is suitable for the treatment of schizophrenia, acute mania, depression, anxiety, bipolar disorder, and mixed episodes associated with bipolar disorder.

The invention is now illustrated in the following examples which are not to be constructed as being limiting.

### EXAMPLES

The following commercially available excipients were used:
microcrystalline cellulose (Avicel PH 102) was purchased from FMC Biopolymer
silicified microcrystalline cellulose (Prosolv HD 90) was purchased from JRS Pharma
colloidal silicon dioxide (Aerosil 200) was purchased from Degussa International
carmellose (NS300) was purchased from Nichrin Chemicals
crospovidone (polyplasdone XL 10) was purchased from ISP Technologies
xylitol (Xylisorb300) was purchased from Roquette
pineapple flavour 501085AP0551 was purchased from Firmenich
aspartame was purchased from Nutrasweet
acesulfame potassium was purchased from Quingdao PTZ
tartaric acid was purchased from Dr. Paul Lohmann
magnesium stearate was purchased from Peter Greven

The following technical equipment for processing and measuring the tablets was used:
sieves ASTM No. 30 and No. 40
high shear mixer granulator from Kevin Process Technologies (capacity: 2.5 liters)
fluid bed dryer from Retsch (capacity: 5.0 liters)
co-mill with sieve from Gansons Quadro No. 7LI68083746(3962)2005-06
bin blender from R.P. Products (capacity: 5.0 liters)
single rotary compression machine (KMP-8) from Kembert
halogen moisture analyser from Mettler Toledo
friabilator, disintegration apparatus and tablet hardness tester from Erweka

Extract from USP Monograph on Silicified Microcrystalline Cellulose:

Identification

A: Infrared Absorption 197K.

B: Prepare iodinated zinc chloride solution by dissolving 20 g of zinc chloride and 6.5

g of potassium iodide in 10.5 mL of water. Add 0.5 g of iodine, and shake for 15 minutes. Place about 10 mg of Silicified Microcrystalline Cellulose on a watch glass, and disperse in 2 mL of iodinated zinc chloride solution: the substance takes on a violet-blue color.

C: Transfer about 5 mg of residue from the test for Residue on ignition to a platinum crucible, and mix with about 200 mg of anhydrous potassium carbonate. Ignite at a red heat over a burner for about 10 minutes, and cool. Dissolve the melt in 2 mL of freshly distilled water, warming if necessary, and slowly add 2 mL of ammonium molybdate TS to the solution: a deep yellow color is produced.

D: Silica dispersion uniformity test

Conditioned Test Substance

Pass Silicified Microcrystalline Cellulose through an 850-μm sieve, disperse it into a suitable scale blender (Planetary mixer, Turbula T2F mixer, or V-blender), and tumble/mix the test substance for a minimum of 20 minutes to condition the material in preparation.

Procedure

Assemble a sieve stack composed of the following nested sieves: 60-, 80-, 120-, 200-, 325-, and 400-US mesh, plus pan. Tare each sieve to the nearest 0.1 g. Accurately weigh 200.0 g of the Conditioned test substance, and transfer to the top sieve. Agitate the sieve stack on a suitable sieve shaker for 20 minutes. Separate and record the weight of each sieve, including the Conditioned test substance fraction. Determine the Conditioned test substance fraction mass by difference. Analyze a test substance from each sieve fraction following Residue on ignition <281>. Obtain the Residue on ignition (ROI) value in percentage, Pi, for each sieve fraction, excluding any fraction weighing less than 0.5 g. Calculate the average percentage of ROI value, PA, for Pi(i=1-6). Calculate the variance for the sieve fraction, excluding the pan and any fraction weighing less than 0.5 g, by the formula:

$$\frac{{\sum }_{i = 1}^{n} {( {P}_{i} - {P}_{A} )}^{2}}{n - 1}$$

The variance should not exceed 0.02.

Microbial Enumeration Tests <61> and Tests for Specified Microorganisms <62>

The total aerobic microbial count does not exceed 1000 cfu per g, and the total combined molds and yeasts count does not exceed 100 cfu per g.

Conductivity

Shake about 5 g with 40 mL of water for 20 minutes, and centrifuge. Retain the supernatant for use in the pH test. Using an appropriate conductivity meter that has been standardized with a potassium chloride conductivity calibration standard having a conductivity of 100 μS per cm, measure the conductivity of the supernatant after a stable reading is obtained, and measure the conductivity of the water used to prepare the test specimen. The conductivity of the supernatant does not exceed the conductivity of the water by more than 75 μS per cm.

pH <791>: between 5.0 and 7.5 in the supernatant obtained in the Conductivity test.

Loss on drying <731>

Dry it at 105 for 3 hours: it loses not more than 7.0% of its weight, within a percentage range, as specified in the labeling.

Residue on ignition <281>: between 1.8% and 2.2%.

Bulk Density

Use a volumeter that has been fitted with a 10-mesh screen. The volumeter is freestanding of the brass or stainless steel cup, which is calibrated to a capacity of 25.0±0.05 mL, and has an inside diameter of 30.0±2.0 mm. Weigh the empty cup, position it under the chute, and slowly pour the powder from a height of 5.1 cm (2 inches) above the funnel through the volumeter, at a rate suitable to prevent clogging, until the cup overflows. [NOTE—If excessive clogging of the screen occurs, remove the screen.] Level the excess powder, and weigh the filled cup. Calculate the Bulk density by dividing the weight of the powder in the cup by the volume of the cup: the Bulk density is within the labeled specification.

Degree of Polymerization

Transfer 1.3 g of Silicified Microcrystalline Cellulose, accurately weighed to 0.1 mg, to a 125-mL conical flask. Add 25.0 mL of water and 25.0 mL of 1.0 M cupriethylenediamine hydroxide solution. Immediately purge the solution with nitrogen, insert the stopper, and shake on a wrist action shaker or other suitable mechanical shaker until completely dissolved. Transfer an appropriate volume of the solution to a calibrated number 150 Cannon-Fenske, or equivalent, viscometer. Allow the solution to equilibrate at 25±0.1° for not less than 5 minutes. Time the flow between the two marks on the viscometer, and record the flow time, t1, in seconds.

Calculate the kinematic viscosity, v1, of the Silicified Microcrystalline Cellulose taken by the formula:

t1(k1)

in which k1 is the viscometer constant (see Viscosity <911>). Obtain the flow time, t2, for a 0.5 M cupriethylenediamine hydroxide solution using a number 100 Cannon-Fenske, or equivalent, viscometer. Calculate the kinematic viscosity, v2, of the solvent by the formula:

t2(k2)

in which k2 is the viscometer constant. Determine the relative viscosity, [η]_{ref}, of the Silicified Microcrystalline Cellulose specimen taken by the formula:

v1/v2

Determine the intrinsic viscosity, [η]_{c}, by interpolation, using the Intrinsic Viscosity Table in the Reference Tables section. Calculate the degree of polymerization, P, by the formula:

(95)[η]_{c},{W_{S}[(100−ROI)/100][(100−LOD)/100]}

in which WS is the weight, in g, of the Silicified Microcrystalline Cellulose taken; ROI is the value, in percentage, obtained from the test for Residue on ignition; and LOD is the value, in percentage, obtained from the test for Loss on drying. The degree of polymerization is not greater than 350.

Particle Size Distribution

Where the labeling states the particle size distribution, determine the particle size distribution as directed in a suitable validated procedure.

Water-Soluble Substances

Shake 5.0 g with about 80 mL of water for 10 minutes, and filter with the aid of vacuum through filter paper (Whatman No. 42 or equivalent) into a vacuum flask. Transfer the filtrate to a tared beaker, evaporate to dryness without charring, dry at 105 for 1 hour, cool in a desiccator, and weigh: the difference between the weight of the residue and the weight obtained from a blank determination does not exceed 12.5 mg (0.25%).

Ether-Soluble Substances

Place 10.0 g in a chromatographic column having an internal diameter of about 20 mm, and pass 50 mL of peroxide-free ether through the column. Evaporate the eluate to dryness in a previously dried and tared evaporating dish with the aid of a current of air in a fume hood. After all of the ether has evaporated, dry the residue at 105 for 30 minutes, cool in a desiccator, and weigh: the difference between the weight of the residue and the weight obtained from a blank determination does not exceed 5.0 mg (0.05%).

Heavy metals, Method II <231>: not more than 0.001%.

### Example 1

TABLE 1

| | | | | |
|---|---|---|---|---|
| | | | Amount | |
| | | Amount | (mg/ | Functional |
| No. | Ingredient | (%) | tablet) | Category |

Intragranulation

| | | | | |
|---|---|---|---|---|
| 1 | Aripiprazole (Anhydrous) | 6.67 | 10.00 | Active Ingredient |
| 2 | Microcrystalline Cellulose | 27.17 | 40.75 | Filler/Diluent |
| 3 | Silicified Microcrystalline | 36.67 | 55.00 | Multiple |
| | Cellulose | | | Functions |
| 4 | Colloidal Silicon Dioxide | 2.00 | 3.00 | Distributing |
| | | | | Angent/Glidant |
| 5 | Purified Water | Q.S. | Q.S. | Solvent |

Extragranulation

| | | | | |
|---|---|---|---|---|
| 6 | Carmellose | 13.33 | 20.00 | Disintegrant |
| 7 | Crospovidone | 3.33 | 5.00 | Disintegrant |
| 8 | Xylitol | 3.33 | 5.00 | Sweetening Agent |
| 9 | Aspartame | 2.00 | 3.00 | Sweetening Agent |
| 10 | Acesulfame Potassium | 2.00 | 3.00 | Sweetening Agent |
| 11 | Tartaric Acid | 2.00 | 3.00 | Flavor Enhancer |
| 12 | Pineapple Flavor | 0.50 | 0.75 | Flavor |
| 13 | Magnesium Stearate | 1.00 | 1.50 | Lubricant |
| | Total | 100.00 | 150.00 | |

The ingredients of Table 1 have been processed as follows:
a) Aripiprazole and Silicified microcrystalline cellulose (SMC) were mixed and sieved through an ASTM No. 40 sieve.
b) Colloidal silicon dioxide and microcrystalline cellulose were mixed and sieved through an ASTM No. 40 sieve.
c) The blends obtained in a) and b) were mixed in a high shear mixer granulator (HSMG).
d) The mixture obtained in c) was subjected to wet granulation with purified water.
e) The material obtained in d) was dried at 80° C. for 25 minutes.
f) The material obtained in e) was milled and sieved through an ASTM No. 30 sieve.
g) The excipients from no. 6 to 12 were co-sifted through an ASTM No. 40 sieve.
h) The excipient no. 13 (magnesium stearate) was sieved through an ASTM No. 40 sieve.
i) The co-sifted excipients obtained in g) were blended with the material obtained in step f).
j) The material obtained in i) was lubricated with sifted magnesium stearate obtained in h).
k) The lubricated blend obtained in j) was compressed to a tablet by using 7.1 mm Flat Faced Bevel Edged punches with an average weight of 150.0 mg/tablet.

Tablet Properties:

| | |
|---|---|
| Average weight of the tablet | 150.0 mg |
| Hardness of tablet | 47N-53N |
| Disintegration time of tablet | 10.0 seconds |

### Example 2

TABLE 2

| | | | | |
|---|---|---|---|---|
| | | | Amount | |
| | | Amount | (mg/ | Functional |
| No. | Ingredient | (%) | tablet) | Category |

Intragranulation

| | | | | |
|---|---|---|---|---|
| 1 | Aripiprazol (Anhydrous) | 6.67 | 10.00 | Active Ingredient |
| 2 | Microcrystalline Cellulose | 25.83 | 38.75 | Filler/Diluent |
| 3 | Silicified Microcrystalline | 36.67 | 55.00 | Multiple |
| | Cellulose | | | Functions |
| 4 | Colloidal Silicon Dioxide | 2.00 | 3.00 | Distributing |
| | | | | Angent/Glidant |
| 5 | Hydroxypropyl Cellulose | 1.33 | 2.00 | Binder |
| 6 | Purified Water | Q.S. | Q.S. | Solvent |

Extragranulation

| | | | | |
|---|---|---|---|---|
| 7 | Carmellose | 13.33 | 20.00 | Disintegrant |
| 8 | Crospovidone | 3.33 | 5.00 | Disintegrant |
| 9 | Xylitol | 3.33 | 5.00 | Sweetening Agent |
| 10 | Aspartame | 2.00 | 3.00 | Sweetening Agent |
| 11 | Acesulfame Potassium | 2.00 | 3.00 | Sweetening Agent |
| 12 | Tartaric Acid | 2.00 | 3.00 | Flavor Enhancer |
| 13 | Pineapple Flavor | 0.50 | 0.75 | Flavor |
| 14 | Magnesium Stearate | 1.00 | 1.50 | Lubricant |
| | Total | 100.00 | 150.00 | |

The ingredients given in Table 2 have been processed as follows:
a) Aripiprazole and Silicified microcrystalline cellulose (SMC) were mixed and sieved through an ASTM No. 40 sieve.
b) Colloidal silicon dioxide and microcrystalline cellulose were mixed and sieved through an ASTM No. 40 sieve.
c) The blends obtained in a) and b) were mixed in a high shear mixer granulator (HSMG).
d) Hydroxypropylcellulose was dissolved in purified water under continuous stirring followed by further stirring for one hour. This binder solution is then used for wet granulation of the mixture obtained in c).
e) The material obtained in d) was dried at 80° C. for 25 minutes.
f) The material obtained in e) was milled and sieved through an ASTM No. 30 sieve.
g) The excipients from no. 7 to 13 were co-sifted through an ASTM No. 40 sieve.
h) The excipient no. 14 (magnesium stearate) was sieved through an ASTM No. 40 sieve.
i) The co-sifted excipients obtained in g) were blended with the material obtained in step f).
j) The material obtained in i) was lubricated with sifted magnesium stearate obtained in h).
k) The lubricated blend obtained in j) was compressed to a tablet by using 7.1 mm Flat Faced Bevel Edged punches with an average weight of 150.0 mg/tablet.

Tablet Properties:

| | |
|---|---|
| Average weight of the tablet | 150.0 mg |
| Hardness of tablet | 51N-59N |
| Disintegration time of tablet | 12.0 seconds |

### Example 3

The ingredients given in table 2 have been processed as follows:
a) Aripiprazole and Silicified microcrystalline cellulose (SMC) were mixed and sieved through an ASTM No. 40 sieve.
b) Colloidal silicon dioxide and microcrystalline cellulose were mixed and sieved through an ASTM No. 40 sieve.
c) Hydroxypropylcellulose was sieved through an ASTM no. 40 sieve.
d) The materials obtained in a), b) and c) were mixed together in a HSMG and the resulting mixture was wet granulated with purified water.
e) The material obtained in d) was dried at 80° C. for 25 minutes.
f) The material obtained in e) was milled and sieved through an ASTM no. 30 sieve.
g) The excipients from no. 7 to 13 were co-sifted through an ASTM no. 40 sieve.
h) The excipient no. 14 (magnesium stearate) was sieved through an ASTM no. 40 sieve.
i) The co-sifted excipients obtained in g) were blended with the material obtained in step f).
j) The material obtained in i) was lubricated with sifted magnesium stearate obtained in h).
k) The lubricated blend obtained in j) was compressed to a tablet by using 7.1 mm Flat Faced Bevel Edged punches with an average weight of 150.0 mg/tablet.

### Example 4

Two different tablets have been prepared by the same process to compare the physical properties thereof. The first one which is named ARIP/68 is a tablet according to the present invention and the second, ARIP/74, contains calcium trisilicate as filler instead of SMCC. All other ingredients and their amounts, however, are the same.

TABLE 3

| | | Batch No.: | | | |
|---|---|---|---|---|---|
| | | ARIP/68 | | ARIP/74 | |
| | | Batch Size: | | | |
| | | 3000 tablets | | 1000 tablets | |
| | | Brief Details: | | | |
| | | | | Composition and | |
| | | Composition and | | Procedure (wet | |
| | | Procedure | | granulation) | |
| | | (wet granulation) of | | of Aripiprazole | |
| | | Aripiprazole ODT | | ODT 10 mg with | |
| | | 10 mg with SMC | | Calcium Trisilicate | |
| | | (Prosolv HD 90) | | instead of SMC | |
| | | Amount | Amount for | Amount | Amount for |
| | | (mg/ | 3000 tablets | (mg/ | 1000 tablets |
| No. | Ingredient | tablet) | (g) | tablet) | (g) |

Intragranulation

| | | | | | |
|---|---|---|---|---|---|
| 1 | Aripiprazole | 10.00 | 30.00 | 10.00 | 10.00 |
| | (Anhydrous) | | | | |
| 2 | Microcrystalline | 38.75 | 116.25 | 38.75 | 38.75 |
| | Cellulose | | | | |
| 3 | Silicified | 55.00 | 165.00 | 0.00 | 0.00 |
| | Microcrystalline | | | | |
| | Cellulose | | | | |
| 4 | Calcium | 0.00 | 0.00 | 55.00 | 55.00 |
| | Trisilicate | | | | |
| 5 | Colloidal Silicon | 3.00 | 3.00 | 3.00 | 3.00 |
| | Dioxide | | | | |
| 6 | Hydroxypropyl | 2.00 | 2.00 | 2.00 | 2.00 |
| | Cellulose | | | | |
| 7 | Purified Water | Q.S. | Q.S. | Q.S. | Q.S. |

Extragranulation

| | | | | | |
|---|---|---|---|---|---|
| 8 | Carmellose | 20.00 | 60.00 | 20.00 | 20.00 |
| 9 | Crospovidone | 5.00 | 15.00 | 5.00 | 5.00 |
| 10 | Xylitol | 5.00 | 15.00 | 5.00 | 5.00 |
| 11 | Aspartame | 3.00 | 9.00 | 3.00 | 3.00 |
| 12 | Acesulfame | 3.00 | 9.00 | 3.00 | 3.00 |
| | Potassium | | | | |
| 13 | Tartaric Acid | 3.00 | 9.00 | 3.00 | 3.00 |
| 14 | Pineapple Flavor | 0.75 | 2.25 | 0.75 | 0.75 |
| 15 | Magnesium | 1.50 | 4.50 | 1.50 | 1.50 |
| | Stearate | | | | |
| | Total | 150.00 | 450.00 | 150.00 | 150.00 |

The batches given in Table 3 above were processed according to the procedure described in example 2, i.e. wet granulating the ingredients no. 1-5 with the binder solution (aqueous hydroxypropyl cellulose solution).

Surprisingly the tablets according to the present invention (batch ARIP/68) show a better performance in disintegration time being 10-12 seconds. The disintegration time of the tablets of batch ARIP/74 was 25-34 seconds.

It was also observed that the weight variation of the tablets ARIP/68 was within acceptable limits during compression whereas in the tablets ARIP/74 a high weight variation of 150.00 mg±7.5% w/w (138.75 mg to 161.25 mg) was observed.

Furthermore, it was observed that the tablets of ARIP/68 were of an optimum hardness of about 50-60 N whereas the tablets of ARIP/74 broke already after applying minor manual forces. Although the tablets of ARIP/74 are compressed with a much higher force, i.e. 95-147 N, the stability of the tablets is poor. This is fact is probably due to the fluffiness of the substance calcium trisilicate which has not an optimal compressibility.

### Example 5

### REFERENCE EXAMPLE

The hardness of three tablets “Abilify® 10 mg Orodispersible tablet” was determined with an Erweka hardness tester according to Pharm. Eur. 6.0 <2.9.8> except that only three tablets were tested. The results are summarized in the following table 4:

| Tablet No. | Hardness (N) |
|---|---|
| 1 | 15 |
| 2 | 14 |
| 3 | 17 |

1. An orally disintegrating pharmaceutical dosage form comprising aripiprazole and 10-70% by weight of silicified microcrystalline cellulose (SMC) based on the total weight of the dosage form.

2. An orally disintegrating pharmaceutical dosage form according to claim 1, further comprising 10-50% by weight of a filler, 0.5-5% by weight of a binder, 5-35% by weight of a disintegrant and 0.5-5% by weight of a distributing agent, based on the total weight of the dosage form.

3. The orally disintegrating pharmaceutical dosage form according to claim 1, comprising 20-65% by weight of SMC, 15-40% by weight of a filler, 1-3% by weight of a binder, 8-30% by weight of a disintegrant and 1-4% by weight of a distributing agent based on the total weight of the dosage form.

4. The orally disintegrating pharmaceutical dosage form according to claim 2, wherein the filler is selected from the group consisting of microcrystalline cellulose, starch, lactose, mannitol and combinations thereof.

5. The orally disintegrating pharmaceutical dosage form according to claim 2, wherein the binder is selected from hydroxypropyl cellulose, ethyl cellulose and combinations thereof.

6. The orally disintegrating pharmaceutical dosage form according to claim 2, wherein the disintegrant is selected from the group consisting of crospovidone, carmellose, croscarmellose sodium, sodium starch glycolate and combinations thereof.

7. The orally disintegrating pharmaceutical dosage form according to claim 2, wherein the distributing agent is selected from the group consisting of colloidal SiO₂, fumed silica, diatomaceous earth, kaolin, talc, magnesium aluminum trisilicate and combinations thereof.

8. The orally disintegrating pharmaceutical dosage form according to claim 2, wherein the filler is microcrystalline cellulose, the binder is hydroxypropyl cellulose, the disintegrant is crospovidone and/or Carmellose, and the distributing agent is colloidal SiO₂.

9. The orally disintegrating pharmaceutical dosage form according to claim 1, wherein said dosage form comprise a tablet.

10. Orally The orally disintegrating pharmaceutical dosage form according to claim 1, wherein aripiprazole is present in an amount in the range of 2-30% based on the total weight of the dosage form.

11. The orally disintegrating pharmaceutical dosage form according to claim 1, wherein the aripiprazole is present in the form of anhydrous aripiprazole.

12. The orally disintegrating pharmaceutical dosage form according to claim 1, wherein the aripiprazole is present in the form of hygroscopic aripiprazole.

13. The orally disintegrating pharmaceutical dosage form according to claim 1, wherein said dosage form has an in vivo disintegration time of 25 seconds or less, as determined by the USP disintegration test method set forth in USP 29, <701> Disintegration, pp. 2670-2672.

14. An orally disintegrating pharmaceutical dosage form comprising aripiprazole, wherein said dosage form has an in vivo disintegration time of 20 seconds or less, as determined by the USP disintergration test method set forth in USP 29, <701> Disintegration, pp. 2670-2672.

15. A process for the manufacture of an orally disintegrating pharmaceutical dosage form containing aripiprazole, said process comprising the steps of
i) blending aripiprazole with silicified microcrystalline cellulose (SMC) and at least one further excipient,
ii) granulating the blend obtained in step i),
iii) blending the granules with at least one further excipient comprising a disintegrant, and
iv) compressing the blend into a tablet.

16. The process according to claim 15, comprising the steps of dry blending aripiprazole with SMC, dry blending a filler with a distributing agent, wet granulating said blends together with a binder solution of hydroxypropyl cellulose in water, drying above 70° C. and adding at least one further excipient comprising a disintegrant.

17. An orally disintegrating pharmaceutical dosage form obtained by the process according to claim 15.

18. (canceled)

19. (canceled)

20. The orally disintegrating pharmaceutical dosage form according to claim 1, wherein said dosage form is formulated with an effective amount aripiprazole suitable for treatment of schizophrenia, acute mania, depression, anxiety, bipolar disorder and/or mixed episodes associated with bipolar disorder.
